(19) United States
(12) Patent Application Publication
(10) Pub. No.: US 2016/0330969 A1
(43) Pub. Date: Nov. 17, 2016

(54) ANTIMICROBIAL COMPOSITIONS AND ARTICLES

(71) Applicant: John F. O'Connell, JR., Framingham, MA (US)

(72) Inventor: John F. O'Connell, JR., Framingham, MA (US)

(21) Appl. No.: 15/107,918

(22) PCT Filed: Oct. 23, 2014
(86) PCT No.: PCT/US14/62072

§ 371 Date: Jun. 23, 2016

Related U.S. Application Data
(60) Provisional application No. 61/894,813, filed on Oct. 23, 2013.

(51) Int. Cl. A01N 59/00; B65D 75/26; A61K 9/00; A01N 25/08; A61K 33/20

(52) U.S. Cl. CPC A01N 59/00; A01N 25/08; A61K 33/20; A61K 9/0014; B65D 75/26

(57) ABSTRACT

The invention provides antimicrobial cleansing compositions comprising hypochlorous acid, in the pH range 2.5 to 6.0, and disposable wiping articles impregnated with these compositions, along with chlorine-impermeable and chlorine-resistant packaging for the compositions and articles. The invention also provides methods of treating blepharitis and acne, and of disinfecting the skin, with these compositions and articles.

### RELATED APPLICATION

This application claims benefit of priority of U.S. Provisional Application No. 61/894,813, filed Oct. 23, 2013, the contents of which are incorporated herein by reference in their entirety.

### FIELD OF THE INVENTION

The invention relates to antimicrobial compositions containing hypochlorous acid, and devices and methods for the use of such compositions.

### BACKGROUND

The utility of alkaline solutions of hypochlorite as disinfecting and sanitizing agents has long been known, and put to common use in such applications as swimming pool sanitization with calcium hypochlorite, and surface and fabric disinfection with sodium hypochlorite (household bleach.) Hypochlorite-based disinfectants are widely used in hospitals, and in the food manufacturing and food service industries. Such compositions are stable to long-term storage, and highly effective, but their extremely high pH renders them corrosive to skin and tissue, and unsuitable for internal or even topical medical applications.

Lowering the pH of a hypochorite solution, to render it less corrosive and suitable for use on human skin and tissue, is readily accomplished by acidification, buffering, or manufacture at or near the desired pH. The classic example is Dakins' solution, a solution of sodium hypochlorite buffered to pH 10, which has a century-long history of use as a topical antiseptic and anti-infective.

The conjugate acid of hypochlorite, hypochlorous acid, is even more effective as a disinfectant. It is generally believed that the notable efficacy of hypochlorous acid, relative to the hypochlorite anion, is due to the molecule's small size and lack of electrical charge, which together permit HOCl to diffuse unimpeded through cell walls and membranes. Bacterial resistance is low, due to the indiscriminate nature of the damage done to the bacterial proteins by HOCl (Prütz, W. A.; Hypochlorous acid interactions with thiols, nucleotides, DNA, and other biological substrates. Arch. Biochem. Biophys. 1996, 332, 110-120). The small size and neutral character also mean that bacterial transport proteins and ion pumps are not effective at pumping HOCl out of the cell. Through the process of evolution, nature has hit on this very solution: neutrophils produce HOCl as part of the anti-microbial immune response. (Albrich, J. M.; McCarthy, C. A.; Hurst, J. K.; Biological reactivity of hypochlorous acid: Implications for microbicidal mechanisms of leukocyte myeloperoxidase. Proc. Natl. Acad. Sci. 1981, 78, 210-214; McKenna, S. M.; Davies, K. J.; The inhibition of bacterial growth by hypochlorous acid. Possible role in the bactericidal activity of phagocytes. Biochem. J. 1988, 254, 685-692.)

Aqueous HOCl solutions, at a sufficiently low pH (4.0-6.0) to provide neutral HOCl as the predominant active species, are therefore very useful and effective topical disinfectants. Uses for such solutions in human health include the treatment of periodontal disease, wound irrigation, instrument disinfection and environmental decontamination. Within the food industry, applications include disinfection of food processing equipment and direct washing of fruits and vegetables, fish, poultry, and meat. Despite decades of effort, however, the production of commercially successful products has proven to be an elusive goal. The difficulty lies in the fact that, while the hypochlorite anion is a stable species in an alkaline solution, the neutral molecule HOCl has very different chemical properties, which render neutral and acidic solutions difficult to store.

The difficulties and limits on HOCl solution stability and storage have been extensively studied: see for example Robinson, G. et al.; The Effect of Long-Term Storage on the Physiochemical and Bactericidal Properties of Electrochemically Activated Solutions. Int. J. Mol. Sci. 2013, 14, 457-469; Len, S. V. et al., Effects of storage conditions and ph on chlorine loss in electrolyzed oxidizing (EO) water. J. Agric. Food Chem. 2002, 50, 209-212; Kunigk, L. et al.; Hypochlorous acid loss from neutral electrolyzed water and sodium hypochlorite solutions upon storage. Braz. J. Food Technol. 2008, 11, 153-158; Hsu, S.-Y. et al.; Effects of storage conditions on chemical and physical properties of electrolyzed oxidizing water. J. Food Engin. 2004, 65, 465-471; and Cui, X. et al.; Physicochemical properties and bactericidal efficiency of neutral and acidic electrolyzed water under different storage conditions. J. Food Eng. 2009, 91, 582-586.

Specifically, the following equilibrium is established at neutral or acidic pH:

HOCl+Cl⁻+H⁺⇄H₂O+Cl₂ (Eqn. 1)

The sanitization of municipal water supplies, by dissolution of elemental chlorine in water, is effective largely because of the formation of hypochlorous acid in the above equilibrium. However, from the viewpoint of storage of hypochlorous acid, the presence of an equilibrium amount of elemental chlorine is a significant problem. Chlorine is very volatile, and readily escapes into the atmosphere, or into the headspace of containers containing solutions of HOCl. It is also a small, neutral molecule, and diffuses readily through the low-density polymers commonly used in liquid packaging. These two factors result in the gradual loss of chlorine from stored solutions of HOCl. Finally, elemental chlorine is highly reactive, and is particularly corrosive in the presence of moisture. Aqueous solutions containing chlorine can therefore defeat ordinary barrier packaging that relies on metal foils, and/or chlorine-reactive high-density polymers, to prevent gas diffusion.

Even if chlorine loss is entirely prevented, HOCl is subject to complex processes by which it decomposes (to chlorite and chlorate species) at neutral pH, with a maximum rate of decomposition at pH 6.89 (Adam, L. C.; et al.; Hypochlorous acid decomposition in the pH 5-8 region. Inorg. Chem., 1992, 31, 3534-3541.) Such processes are catalyzed by light, therefore solutions of HOCl also require low-actinic packaging.

The remarkable utility of HOCl solutions, and the remarkable difficulty of storing them, has created a market for machinery capable of generating such solutions in situ. In general, these devices operate by the electrolysis of chloride-containing water, which can be done crudely in a non-membrane cell, but is more usually carried out in a continuous flow membrane cell system. At the anode, chloride is oxidized to yield hypochlorous acid:

Cl⁻+H₂O→HOCl+H⁺+2e⁻ (Eqn. 2)

The freed hydrogen ions render the anode effluent acidic. Hydrogen is evolved at the cathode, where the attendant consumption of hydrogen ions creates an alkaline solution. The various commercial systems use the acidic “anode water” directly, or combine it with the alkaline cathode solution and/or added buffers, to yield a solution of HOCl having a desired pH. See, e.g., U.S. Pat. Nos. 5,858,201, 6,793,846 and 7,749,370.

The products of these systems are promoted under a variety of names, such as “High ORP” (Oxidation Reduction Potential) water, “NOW” (Neutral Oxidized Water), “SOW” (Super Oxidized Water), “ECAS” (Electrochemically Activated Solutions), “SAEW” (Slightly Acidic Electrolyzed Water), “EO” (Electro-Oxidized) or “acidic EO” water, and even “nanoclustered” water.

All such prior art HOCl solutions will be referred to herein as FAC (Free Available Chlorine) solutions or FAC water. FAC solutions typically have a pH between 5 and 7, depending on the feed solution, electrode materials and any blending with cathode water. FAC solutions may contain oxidative species in addition to HOCl (e.g., ozone, dissolved oxygen and hydrogen peroxide), which purportedly enhance their antimicrobial properties.

FAC solutions have shown wide utility as disinfectants in hospital and food processing facilities, and are promoted as skin cleansing and wound treatment therapies, because of their rapid and broad-spectrum antimicrobial activity against a wide range of bacteria, viruses, and spores. FAC solutions are surprisingly well-tolerated by mammalian cells, and they are environmentally benign, avoiding the need for costly disposal procedures. As noted above, FAC solutions do not promote microbial resistance or tolerance, which is of particular value in hospital applications.

Although standard FAC solutions are effective disinfectants, they generally have a limited shelf life (a few hours to a few days) due to the reactivity of the HOCl itself, the presence of chloride ions, which displace Equation 1 to the right, and the volatility and reactivity of the elemental chlorine thus produced, the loss of which continues to drive Equation 1 to the right. Any reduction of HOCl and/or chlorine by oxidizable contaminants or container materials produces additional chloride, further contributing to the loss of active chlorine.

The nonselective reactivity of FAC water means that the solution can be inactivated by the oxidizable organic load present at the site of application, so that high volumes of FAC water may be needed to obtain effective disinfection in applications such as floor washing and food processing. Transportation of FAC solutions in large quantities, however, requires compliance with applicable transportation regulations, which can be a costly nuisance. As a result of the short lifespan and, in some cases, the need for high volumes, economical production of FAC water generally must take place in close proximity to where the FAC water is to be used. The result is that hospitals, clinics, agricultural producers and food processors must purchase, house and maintain the equipment necessary to produce FAC water, which represents significant capital and operating expenses.

Accordingly, there has long been a need for topical disinfectants that have the advantages of HOCl, while addressing the shortcomings of the electrolytic FAC solutions discussed above. Products described as “stabilized” HOCl, such as the MICROCYN™ and DERMACYN™ solutions marketed by Oculus Innovative Sciences (see Landa-Solis, C. et al.; Microcyn: a novel super-oxidized water with neutral pH and disinfectant activity. J. Hosp. Infect. 2005, 291-299), STERILOX™ solutions marketed by PuriCore Plc., and NeutroPhase™ solution manufactured by NovaBay Inc. and marketed by Principle Business Enterprises, have been introduced into the market. The stabilization of these products appears to be a function of relatively low chloride concentrations, on the order of 0.5-1.0%, and/or a relatively high pH (6.0-7.0), so that the reaction of Equation 1 proceeds at a slower rate. However, even these low concentrations of chloride still equal or exceed the concentration of hypochlorous acid (generally 40-500 ppm, i.e. 0.01-0.05%), so that chlorine is still lost over the course of time. The FAC solution sold under the trade name NIXALL™, for example, is distributed in polypropylene bottles, and per package labeling must be used within 30 days of manufacture. Other products have a shelf life of about 12 months at best, unless stored in all-glass containers, and remain effective for as little as 2-4 weeks once the packaging is opened. Manufacture, storage, and distribution have to be run on a schedule that minimizes inventory, and out-of-date inventory must be discarded, resulting in prices that are only marginally competitive with on-site FAC production. There remains a need for more stable HOCl products.

Blepharitis is a common inflammation of the eyelids that can be caused by a bacterial eye infection, or the inflammation can be associated with a skin condition such as acne rosacea. Another type of blepharitis, demodex blepharitis, is caused by microscopic mites (demodex folliculorum) that clog follicles at the roots of eyelashes. A prevalent form of blepharitis is anterior blepharitis, usually caused by Staphylococcus spp., in particular S. aureus. Symptoms of blepharitis can include a burning sensation of the eyes, itching or inflammation of the eyelids or eyelid margins, crusting, tearing, and irritation or foreign body sensation. The lashes can be powdered with dry scales. Blepharitis can be a chronic condition that is difficult to treat or cure.

Current treatments for blepharitis are regular cleaning of the eyelids with over-the-counter (OTC) eye cleansers as well as the use of antibiotics and sometimes steroids and antihistamines. See, e.g., U.S. Patent application publication Nos. 2012/0015971, 2011/0059925 and 2010/0227842, and U.S. Pat. Nos. 8,349,806 and 6,113,894. However, the current treatments have met with limited success. Due to limits on their ability to rapidly penetrate into the skin, an effective amount of a cream or ointment must be left for a period of time on the eyelids, leading to difficulties with patient compliance. In addition, antibiotics bring the danger of inducing resistance in the target bacteria.

Products specifically designed as “eye scrubs”, which are actually eyelid washing compositions, are available and are preferred over simple hypoallergenic soap or shampoo. Key, J. E.; A comparative study of eyelid cleaning regimens in chronic blepharitis. CLAO Journal, 1996, 22, 209-212; Doan S.; Tolerability and acceptability of Blephagel: a novel eyelid hygiene aqueous gel. Clin. Ophthalmol. 2012, 6, 71-77; Avisar, R. et al; Effect of I-Scrub on signs and symptoms of chronic blepharitis. J. DICP 1991, 25, 359-360.

An aqueous solution of hypochlorous acid has been suggested as a treatment for blepharitis and other infections of the eye (US 2010/0285151 and WO 2008/089268). However, hypochlorous acid is not currently on the market, nor is it approved for marketing, for the treatment of blepharitis. As noted above, the commercial solutions that are available are costly, and their limited shelf life makes it difficult to manage inventory and distribution in the pharmacy business. There remains a need for a safe and effective treatment of blepharitis and related eye conditions caused by microorganisms. The present invention provides such a treatment.

The compositions and methods discussed above are not admitted to be prior art, and should not be regarded as such, merely as a result of being noted in the “background” section. More particularly, the problems discussed above are not admitted to have been previously recognized in the prior art, and should not be regarded as such merely as a result of being noted in the “background” section.

### BRIEF DESCRIPTION OF THE INVENTION

The invention provides skin-cleansing compositions, having a pH from 3.0 to 7.0, comprising water, hypochlorous acid, one or more optional buffers, and optionally a dermatologically-acceptable surfactant that is storage-stable in the presence of hypochlorous acid in the pH range 3.0 to 7.0. The compositions have less than 0.5% w/v chloride, and preferably have a pH from 4.0 to 6.0. The invention also provides disposable wiping articles and bandages impregnated with these compositions. In one aspect of the invention, the above compositions and articles are provided within chlorine-impermeable and chlorine-resistant packaging. The packaging provided by the present invention prolongs the time during which the hypochlorous acid remains at an effective concentration.

The invention provides methods for disinfecting the skin, and cleaning wounds and burns, with the compositions and articles of the invention.

The invention provides a method of treating blepharitis, which comprises scrubbing the affected eyelid with a composition of the invention. In one embodiment, the eyelid is scrubbed with a disposable wiping article impregnated with this composition.

The invention also provides wiping articles impregnated with the compositions of the invention that are suitable for cleaning and disinfecting environmental surfaces in homes, hospitals, and businesses. These wipes are larger than the medical wipes described above, and may incorporate surfactants that are not necessarily dermatologically-acceptable.

### BRIEF DESCRIPTION OF THE FIGURES

FIG. 1 shows the molar percentages of the species HOCl, OCl⁻, Cl₂ (aq.) and Cl₂ (gas) as a function of pH.

FIG. 2 shows the rate of escape of chlorine from jars having different lid liners.

### DETAILED DESCRIPTION OF THE INVENTION

The invention provides skin-cleansing antimicrobial compositions, having a pH from 3.0 to 7.0, comprising water, hypochlorous acid, one or more buffers, and optionally, a dermatologically-acceptable surfactant that is storage-stable in the presence of hypochlorous acid in the pH range 3.0 to 7.0. The compositions preferably have a pH from 4.0 to 6.0, and more preferably from 4.5 to 5.5, and have a concentration of chloride that is less than about 0.5% w/v, preferably less than about 0.1% w/v, and most preferably less than about 0.05% w/v. In particular embodiments, the composition is an eyelid-cleansing composition.

The invention also provides environmental surface-cleansing antimicrobial compositions, having a pH from 3.0 to 7.0, comprising water, hypochlorous acid, one or more buffers, and optionally, a surfactant that is storage-stable in the presence of hypochlorous acid in the pH range 3.0 to 7.0. These compositions also preferably have a pH from 4.0 to 6.0, and more preferably from 4.5 to 5.5, and have a concentration of chloride that is less than about 0.5% w/v, preferably less than about 0.1% w/v, and most preferably less than about 0.05% w/v.

The invention further provides chlorine-impermeable and chlorine-resistant packages, containing the above compositions. In preferred embodiments, the packages have substantially zero headspace. As used herein, “substantially zero headspace” refers to a headspace volume that is less than about 2% of the volume of the package. In certain embodiments, the package is collapsible, is fitted with a movable seal, or is otherwise adapted to retain substantially zero headspace as the composition is dispensed.

In particular embodiments, the chlorine-impermeable and chlorine-resistant package contains an antimicrobial eyelid-cleansing composition, and instructions to cleanse an eyelid with a single dose of the composition. Such a package preferably contains fewer than about 70 doses, and more preferably fewer than about 35 doses.

The invention further provides disposable wiping articles impregnated with the antimicrobial cleansing compositions described above, and adhesive bandages having an absorbent pad, where the pad is impregnated with the antimicrobial cleansing compositions described above.

The invention further provides chlorine-impermeable and chlorine-resistant packages containing the wiping articles and adhesive bandages. These packages may take the form of a jar having a lid. The lid may be chlorine-impermeable, and/or may be provided with a lid liner that is chlorine-impermeable. The lid liner comprises at least one layer of a chlorine-impermeable polymer, and/or comprises a chlorine-impermeable metal-polymer laminate.

In alternative embodiments, the chlorine-impermeable and chlorine-resistant package is a flexible pouch, which may be of a tear-open design, and which may be re-sealable. Suitable designs for tear-open and re-sealable pouches are well-known in the art, and any of these are contemplated to be operable in the present invention. It will be appreciated that flexible pouches constitute collapsible containers that are, in general, adapted to retain substantially zero headspace within. Flexible pouches are preferably constructed from a barrier film that comprises at least one chlorine-impermeable polymer layer. Flexible pouches are preferably constructed from laminated films, which may be entirely formed from polymer layers, or which may be formed from polymer-metal laminates.

The invention provides a method of treating an eyelid affected with anterior blepharitis, which comprises the steps of (a) removing a wiping article of the invention from a chlorine-impermeable and chlorine-resistant package, and (b) wiping the eyelid with the wiping article. The invention also provides a method of treating skin affected with acne, which comprises the steps of (a) removing a wiping article of the invention from a chlorine-impermeable and chlorine-resistant package, and (b) wiping the affected skin with the wiping article. Furthermore, the invention provides a method of disinfecting the surface of an area of human skin, comprising the steps of (a) removing a wiping article from the chlorine-impermeable and chlorine-resistant package, and (b) wiping the surface of the area of the skin with the wiping article. The latter method finds utility in preparing a patient for a blood draw or an injection, or for the insertion of a catheter or IV line.

Hypochlorous acid solutions may be manufactured by various means known in the art. Some such methods have been known for many years. For example, a solution or suspension of calcium hypochlorite can be acidified to a pH of between 4 and 6 with an organic or inorganic acid, as taught in U.S. Pat. Nos. 2,111,194 and 2,199,936 and U.S. Patent Application publication No. 2014/0134224. When sulfuric or phosphoric acid are employed, removal of precipitated calcium phosphate or sulfate yields the desired hypochlorous acid solution, substantially free of calcium ions. Continuous processes based on the reaction of chlorine with water and/or alkaline salts have been developed for large-scale synthesis; see e.g. U.S. Pat. Nos. 4,147,761, 5,322,677 and 6,048,513. Electrolytic methods are referenced above.

The loss of elemental chlorine, due to its volatility and reactivity, tends to drive the equilibrium of Equation 1 to the right, leading to the depletion of HOCl over time:

HOCl+Cl⁻+H⁺⇄H₂O+Cl₂↑ (Eqn. 1)

A low concentration of chloride, however, displaces the equilibrium reaction of Eqn. 1 to the left. The shift to the left, and the slowing of the forward reaction, together serve to substantially prevent the loss of hypochlorous acid via this mechanism. The use of low-chloride HOCl solutions in the compositions of the invention, accordingly, provides products that are more stable to storage. For this reason, preferred methods of manufacture are those that produce low-chloride solutions of hypochlorous acid. In preferred embodiments, the compositions of the invention have less than 0.5% w/v chloride, and more preferably less than 0.05%. Most preferably, the chloride content is less than 0.01% w/v.

Electodialysis can be used to remove the bulk (ca. 90-94%) of chloride ions from a solution of HOCl, as described in U.S. Pat. No. 3,616,385. The efficiency of chloride removal by electrodialysis is limited, however, by the drop in conductivity as the concentration of ions in the solution drops. If desired, conductivity can be restored, and the removal of residual chloride can be improved, by the addition of inert anions such as acetate, phosphate and sulfate.

Alternatively, chloride ions may be removed by passage of the HOCl solution through an anion exchange resin, exchanging chloride for hydroxide, phosphate, sulfate, acetate, or other inert anions. This has been described as the second step of a process employing the sequential use of cation and anion exchange resins, to remove both metal and halide ions from hypochlorite solutions, so as to generate salt-free HOCl solutions in the pH range of 3.5-7.5 (see Japanese Kokai publication No. 2009-274950.) Suitable resins for such processes include those marketed under the trade names DOWEX™ (Dow Chemical Co., Midland, Mich.) and AMBERLITE™ (Rohm and Haas Co., Philadelphia Pa.).

Japanese Kokai publication No. H06-206076 describes the use of cation-exchange resins to prepare low-pH, cation-free solutions of HOCl. The difficulty with this process is that the exchange of all metal ions for hydrogen ions leads to very acidic solutions of HOCl, which is unstable toward decomposition to elemental chlorine via Eqn. 1. For this reason, the authors were obliged to raise the pH to at least 6.0, and to this end they added alkaline HOCl solution, containing chloride, to the column effluent. To avoid the generation of strongly acidic solutions, weakly acidic cation exchange resins, e.g. AMBERLITE™ IRC86, are preferred in this process, as disclosed in European patent application publication No. EP 2565156.

To the inventor's knowledge, the use of an anion-exchange resin to prepare halide-free HOCl, without first removing the metal cations, has not been reported. This is undoubtedly due to the fact that alkalinization would ionize the HOCl, and the resin would retain the resulting hypochlorite ions.

It is well-known that mixed-bed ion-exchange resins, in OH⁻/H⁺ form, are effective in removing trace amounts of both cations and anions. Aqueous HOCl, at pH 4-6, is largely un-ionized, and would be expected to elute from a column of a mixed-bed resin in substantially de-salted form. Due to the low concentration of HOCl in the compositions of the invention, however, most commerically-available resins require pre-treatment with an acidified (pH 5) hypochlorite solution to remove oxidizable impurities. This renders mixed-bed resins unsuitable for the present application, because they are not readily regenerated to the H+/OH− form after such a pre-treatment; this is the reason separate anion- and cation-exchange steps are employed in the above-cited prior art methods.

The inventor has discovered, however, that an amphoteric ion exchange resin (e.g., DOWEX™ RETARDION™ 11A8), which is regenerable by elution with hot water (ca. 65° C.), is suitable for single-bed desalting of HOCl solutions. However, the use of this or any other ion-exchange resin to desalt electrolytically-produced HOCl is inefficient, in that the relatively high concentration of sodium chloride rapidly exhausts the capacity of the resin. For example, a column with a bed volume of one liter would have the capacity to absorb roughly one mole (58 g) of sodium chloride, corresponding to only one liter of 5.8% NaCl solution, or two liters of 2.9% NaCl. Efficient use of ion exchange resins requires that fifty or more column volumes of solution be processed before regeneration of the resin becomes necessary. Accordingly, the present invention provides a process in which the use of an amphoteric resin is coupled with a low-chloride method of synthesis of HOCl, whereby one volume of resin is sufficient to desalt 900-1,000 volumes of an HOCl solution.

A suitable method is the acidification of a purified form of calcium hypochlorite, having 75-78% available chlorine, which has recently become commercially available. In this material, calcium chloride and sodium chloride, combined, are in the range of 0-8%. A suitable example is the material marketed under the trade names POOLIFE TURBOSHOCK™ (Lonza Group Ltd, Basel, Switzerland) and POWER SHOCK™ (Arch Chemicals Inc., Atlanta Ga.) Preferred acidification reagents are sulfuric acid and phosphoric acid.

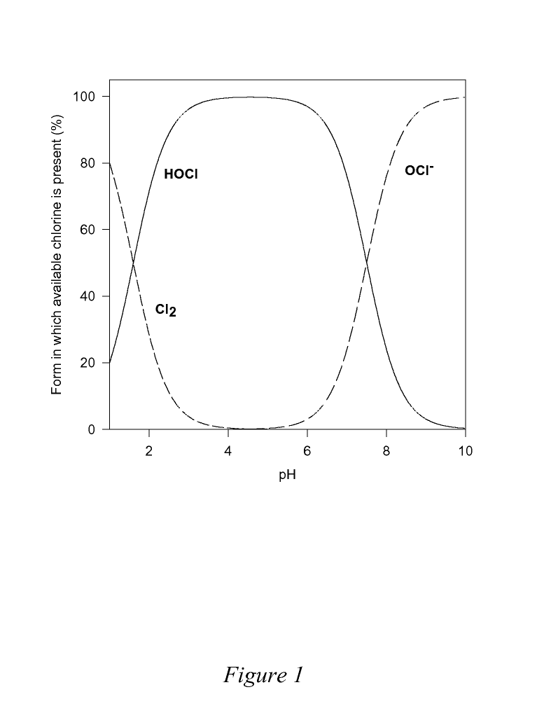

The compositions of the invention may contain between 5 and 1000 mg HOCl per liter, and preferably contain between 15 and 500 mg per liter. More preferred embodiments contain between 50 and 300 mg per liter. The HOCl solutions of the invention may be modified by the addition of one or more buffers. As can be seen by inspection of FIG. 1, the concentration of HOCl relative to other species reaches a maximum at about pH 5.0. (FIG. 1 presents a first-order approximation; more detailed calculations, taking into account all significant equilibria in such solutions, are disclosed in U.S. Patent Application publication No. 2014/0134224.) Since the active antimicrobial agent is HOCl, maximum effectiveness will be associated with a maximum concentration of this species, and the preferred buffers will stabilize the pH at a value between 3.0 and 7.0, more preferably between 4.0 and 6.0, and most preferably between 4.5 and 5.5. The practitioner may choose to vary the pH within these ranges in order to optimize other properties, such as storage stability or cleaning efficiency. Suitable buffers include, but are not limited to, phosphate and acetate. Phosphate has poor buffering capacity at pH 5.0, however, and acetate is much preferred.

The elemental chlorine created according to Eqn. 1, if kept in solution and prevented from escaping, will maintain the equilibrium and prevent the reaction from proceeding to the right. For this reason, in another aspect of the invention, chlorine-impermeable and chlorine-resistant packaging is provided for the compositions of the invention, and one aspect of the invention provides the above compositions contained within such packaging. The compositions within such packaging are preferably provided with zero, or substantially zero, headspace, so as to minimize the volume of gas into which chlorine may escape from solution during storage.

In certain embodiments, the packaging of the invention is adapted for the drop-wise dispensation of the composition; for example when the composition is intended to be used as eye drops. In certain embodiments of such packaging, the packaging is adapted to preserve a condition of zero or substantially zero headspace, even as the composition is dispensed. Such packaging may, for example, take the form of a syringe having a plunger, or a collapsible container formed from a deformable material, which is adapted to dispense the composition when the package is squeezed. Examples include, but are not limited to, collapsible tubes, ampoules, and sachets. The package is preferably provided with a dropper tip for instilling the composition dropwise into the eye. A one-way valve may be incorporated into the dispensing device, so as to prevent air from entering the container and creating a headspace.

Although it is desirable to maintain a small headspace within the container during storage, maintenance of this small headspace after the container is opened (and dispensation of the composition has begun) may not be necessary if the contents are dispensed within an acceptably short period of time. An acceptably short period of time is the time over which the total loss of HOCl is deemed acceptable, for example less than about 10%, 20%, or 30%. The acceptable loss will be a function of the initial HOCl concentration and the minimum effective HOCl concentration for the intended use. Accordingly, in situations where the contents are expected to be dispensed within an acceptably short period of time, typical bottles and vials used for ocular solutions, as well as collapsible tubes, ampoules, and sachets, can be employed without provision for maintaining a small headspace after opening. Suitable containers are well-known in the art; examples may be found in, for example, U.S. Pat. Nos. 7,776,355 and 7,971,755.

In order to accommodate the finite lifetime of the composition after a package is opened, and ensure dispensation within an acceptably short period of time, the packaging may, in certain embodiments, contain a limited number of doses, for example a one-week, two-week, one-month or two-month supply if the composition is used in the recommended manner, or as prescribed. In certain of these embodiments, the packaging may contain fewer than about 150 doses, fewer than about 100 doses, fewer than about 70 doses, or fewer than about 35 doses. In certain embodiments, the packaging may contain as little as one or two doses. Depending on the application, a dose may range from a few drops, if the composition is to be instilled in the eye, to several milliliters if the composition is to be used to moisten a bandage, cotton ball or gauze pad. A dose may be a fluid ounce or more, for example if the composition is to be used for wound irrigation or hand washing. In another aspect of the invention, a wet wiping article is provided, and a dose will consist of a single such article impregnated with a composition of the invention.

Among the materials most suitable for containing the compositions of the invention is glass, which is entirely inert to and impermeable by chlorine. This has been demonstrated with an acidic (pH 3.5) solution of HOCl in torch-sealed glass vials (see U.S. Pat. No. 7,393,522), but such containers are impractical for a consumer product, and the low pH permits chlorine to form in unacceptably high concentration. Low-actinic glass is readily available, which helps prevent photo-induced degradation of hypochlorite. Glass is a preferred material, where the composition is expected to be dispensed within an acceptably short period of time. However, glass containers are rigid, making it difficult to maintain a low headspace once the contents are only partially dispensed. Plunger devices are available for this purpose, and may be employed in the present invention despite their added cost and complexity. Caps for glass bottles and jars containing the compositions of the invention are preferably chlorine-resistant and chlorine impermeable, and may be made from and/or lined with the polymers and films described below.

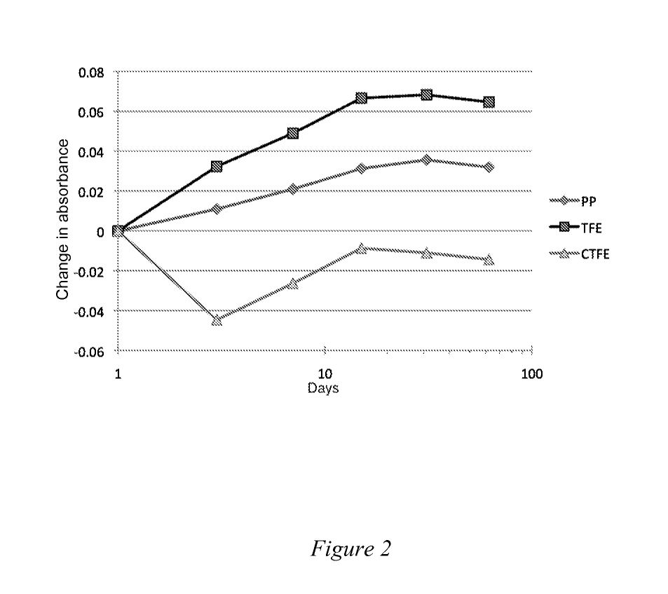

As a rough approximation, the rate of diffusion of chlorine through the cap of a glass container is directly proportional to the area of the cap, and the rate at which the HOCl concentration within a given container decreases is inversely proportional to the volume of the contents. Thus, glass bottles having prior art caps and cap liners are moderately effective at containing chlorine. However, bottles are not suitable for the containment of wipes, which requires jars that permit access to and removal of the wipes. A jar is characterized by having a cap that is essentially of the same diameter as the jar (i.e., within about 10%), and the cap accordingly represents a significant fraction of the total surface area of the container. Loss of chlorine from jars having conventional polypropylene caps takes place at an unacceptably high rate, unless the caps are fitted with chlorine-barrier lid liners, as shown in FIG. 2.

Glass is also fragile, and breakage is especially likely if the package is dropped on the tile flooring typically installed in bathrooms. Accordingly, HOCl compositions contained within rigid but non-fragile plastic jars and bottles constitute another preferred embodiment of the invention. Caps and/or cap liners for plastic jars and bottles containing the compositions of the invention will also preferably be chlorine-resistant and chlorine impermeable. Suitable polymers for forming jars and bottles containing the compositions of the invention are known, for example cyclic olefin polymer (COP) and cyclic olefin copolymer (COC). Polyethylene terephthalate ester (PETE), preferably having gas barrier properties enhanced by incorporation of a nylon-6 nanocomposite, is another suitable polymer. Polymer jars and bottles may optionally be provided with a silica coating, to provide even greater resistance to gas permeability; such containers are commercially available from SiO2 Medical Products Inc, Auburn Ala.

Collapsible containers are preferred in those embodiments of the invention where the contents are not expected to be dispensed within an acceptably short period of time. Examples of multi-layer flexible films, suitable for collapsible containers and having low oxygen permeability, are described in, for example, U.S. Pat. Nos. 8,029,885 and 8,486,501, U.S. Patent Application publication No. 2012/0271270, and in the references contained therein. Films having alternating layers of polymer and clay nanoparticles provide excellent gas barrier properties, and are also suitable for use in the present invention (see, e.g., M. Priolo et al, Nano Lett., 2010, 10 (12), pp 4970-4974.) Preferably the innermost layer is a gas barrier polymer such as LLDPE or oriented PET, selected for its chemical resistance to chlorine and HOCl. This is welded or adhered via a tie layer to the next layer of the film, which may be a polymer or may be a metal foil such as aluminum, which serves as a gas barrier against loss of chlorine. For the greatest level of resistance, this optional foil layer may be formed from silver or gold. As is known in the art, metallic foils in laminated films need only be thick enough to reliably have few or no detectable pinholes, typically 0.005 to 0.01 mm in thickness. The outermost layer may also be a polymer layer, and will typically be chosen for its mechanical strength and/or the ability to accept printing. In one preferred embodiment, the inner layer is LLDPE, and the gas barrier layer is 0.007 mm aluminum foil, adhered by an ethylene-acrylic acid copolymer based tie layer, such as that marketed by Glenroy Inc. (Menomonee Falls, Wis.) under the trade name GRX™. The outer layer may be a printable, coated PET, tied to the aluminum with extruded LDPE. A suitable example of this construction is the laminate sold by Glenroy Inc. with product number EFS 145-001. Applicants have surprisingly found that the aluminum in such a laminate is resistant to degradation by chlorine.

In another aspect of the invention, disposable wiping articles, pre-moistened with an antimicrobial composition of the invention, are provided. The wiping articles may be discs or pads of woven or non-woven hydrophilic fabric, or may be formed from a hydrophilic polymer foam. In another aspect of the invention, chlorine-impermeable and chlorine-resistant packaging is provided for the above articles. Such packaging is preferably provided with zero, or substantially zero, headspace, or is of the collapsible form described above.

The wiping articles, colloquially known as “wet wipes”, may be of the general physical form described in, for example, U.S. Pat. Nos. 4,891,228 and 5,888,524. The antimicrobial wet wipes of the invention may take any of the several different forms know in the art. For example the wet wipes may be in the form of a stack of moistened sheets which have been packaged in a jar or flexible packet. The wipes may be in a folded or unfolded configuration. In addition, the wipes may be in the form of continuous webs of material which include perforations to separate the individual wet wipes from the continuous web. Such continuous webs may be wound into rolls and also packaged in plastic containers. The wipes can be used for baby wipes, hand wipes, household cleaning wipes, industrial wipes, and the like. A particular use is for treating skin infections, such as blepharitis, as described in greater detail below.

Materials suitable for the antimicrobial wet wipe of the present invention are well known to those skilled in the art. The wet wipe can be made from any material suitable for use as a moist wipe, including meltblown, spun-bonded, coform, air-laid, bondedcarded web materials, hydroentangled materials and the like. The wet wipe may have a basis weight of from about 25 to about 120 grams per square meter and desirably from about 40 to about 90 grams per square meter. The wipes are preferably formed from materials that are storage-stable in the presence of chlorine and hypochlorous acid, at the pH of the composition being employed. “Storage-stable”, in this context, means that the wipes remain usable for their purpose for a period of at least 12 months at room temperature. Preferred polymers for storage-stable wipes are free of groups which are subject to chlorination, such as such as olefin, amino, amido NH, and hydroxyl groups.

Suitable articles may be formed, for example, from woven or non-woven wettable polyolefin or polyester fibers, modified with hydrophilic monomers or having a hydrophilic surface treatment. Other polymers suitable for inclusion in the fibers include, but are not limited to, polyacrylic acid (PAAc), polyethylene oxide (PEO), polyethersulfone (PES), and blends and copolymers thereof. Normally hydrophobic polymers may be rendered wettable by methods known in the art, such as are described in U.S. Pat. No. 5,614,574.

By way of example, coform basesheets may comprise a gas-formed matrix of thermoplastic polymeric meltblown microfibers, such as, for example, polypropylene microfibers, and hydrophilic fibers as described above. The relative percentages of the polypropylene microfibers and hydrophilic fibers in the coform basesheet can vary over a wide range depending on the desired characteristics of the wipes. For example, the coform basesheet may comprise from about 20 to about 60 weight percent, and more desirably from about 30 to about 40 weight percent of polypropylene microfibers based on the dry weight of the coform basesheet being used to provide the wipe. Again by way of example, wipes can be made from meltblown or spunbond polyolefin or polyester sheets having a basis weight of from about 25 to about 120 grams per square meter. The preferred sheet materials are woven or non-woven PET, and various wettable variations thereof.

The wet wipes are saturated or otherwise impregnated with an antimicrobial composition of the present invention. The amount of the antimicrobial composition added to the wipes will vary depending upon the type of material being used to provide the wipe, the type of container being used to store the wipes, and the desired end use of the wipes. Generally, each wipe can contain from about 150 to about 600 weight percent and desirably from about 250 to about 450 weight percent of the antimicrobial composition based on the dry weight of the wipe. In a particular aspect wherein the wet wipe is made from a coform material comprising from about 30 to about 40 weight percent polymeric microfibers based on the dry weight of the wipe, the amount of the antimicrobial composition contained within the wet wipe is from about 300 to about 400 weight percent and desirably about 330 weight percent based on the dry weight of the wet wipe. If the amount of liquid is less than the above-identified range, the wet wipe may be too dry to moisten the surface being wiped, and may not adequately perform. In certain embodiments, the wipes are simply saturated with, and optionally immersed in a slight excess of, the HOCl composition, and the container is sealed with zero or substantially zero headspace.

In an alternative embodiment of the invention, the wipes are provided in a container in a dry state, and the consumer is provided with an antimicrobial HOCl solution of the invention in a separate container. The wet wipes of the invention are prepared in situ, by pouring the solution over the wipes. The container for the wipes in this embodiment may have a reduced level of chlorine resistance and impermeability, as the wipes will require containment only until they are used up.

The invention also provides wiping articles contained within a chlorine-impermeable and chlorine-resistant package. In certain of these embodiments, the package may be a glass or polymer jar, as described above. In alternative embodiments, the package may be a tear-open pouch, preferably formed from a laminate as described above.

Tear-open pouches are preferably formed from a polymer, multi-polymer laminate, or polymer-metal laminate. The polymer, or at least one polymer in a laminate, is preferably chlorine-resistant and chlorine-impermeable, and compatible with lamination processes such as coextrusion. There is relatively little data on the chlorine permeability of polymer films, but chlorine, like oxygen, is a small, neutral, diatomic molecule, and is expected to have similar diffusion properties, albeit with a roughly 30% slower diffusion rate (per Graham's Law) due to its greater molecular weight. Thus, in general, polymers and materials that provide oxygen barrier functionality are expected to have chlorine-barrier functionality as well, and—provided that they are unreactive with chlorine—such materials are expected to be useful in the present invention. Oxygen diffusion rates through polymer films can be measured by known methods; see for example Rharbi, Y.; Yekta, A.; Winnik, M. A.; A Method for Measuring Oxygen Diffusion and Oxygen Permeation in Polymer Films Based on Fluorescence Quenching, Anal. Chem. 1999, 71, 5045-5053. Those of skill in the art will be familiar with numerous reference volumes that list oxygen transmission rates for a wide variety of commercially-available polymers. Values for the Oxygen Transmission Rate (OTR) of exemplary polymers are presented in Table 1; the values for the Chlorine Transmission Rate (CTR) are expected to vary similarly and in similar proportions.

The OTR values in Table 1 represent the volume of oxygen (in ml) that will diffuse through 100 square inches of a 1-mil film, over the course of 24 hours at room temperature and atmospheric pressure. Table 2 illustrates the considerable advantages of high-barrier polymers over ordinary polyethylene (LDPE) and polypropylene containers:

TABLE 1
Oxygen barrier comparison of representative polymers

| | OTR @ 25° C. |
|---|---|
| Material | (ml-mil/100 in²-24 hr-atm) |
| Silica-coated PET | 0.002-0.07 |
| EVOH (dry) | 0.01 |
| EVOH (coextruded) | 0.04-1.07 |
| PVdC | 0.07 |
| MXD6 (dry) | 0.15 |
| MXD6 (coextruded) | 0.25 |
| Polyacrylonitrile | 1.0 |
| PEN | 1.2 |
| PET (amorphous) | 1.0 |
| PET (oriented) | 3.0-5.0 |
| Nylon (amorphous) | 1.5-2.4 |
| LDPE | 420 |
| HDPE | 150 |
| Polypropylene | 150 |
| Polystyrene | 350 |

Ethylene vinyl alcohol copolymer (EVOH), polyvinylidene dichloride (PVdC), vinylidene dichloride/vinyl chloride copolymer (SARAN™ film), polyacrylonitrile, polyethylene naphthalate (PEN), metaxylylene nylon-6 (MDX6) and nylon-6 nanocomposite (MXD6-NC) are examples of particularly effective oxygen barrier polymers that may be used in the present invention. Oriented and amorphous polyethylene terephthalate (PET) and oriented and amorphous nylon are also expected to be suitable. Cyclic olefin polymers and copolymers (COP and COC) available from Topas Advanced Polymers (Florence, Ky.) represent another class of similarly suitable materials. Polychlorotrifluoroethylene (CTFE), available under the trade name ACLAR™ (Honeywell International Inc., Morristown, N.J.) is one example of a material having desirable chlorine resistance; another is ETFE, available under the trade name TEFZEL™ (DuPont Inc., Wilmington, Del.). PTFE (TEFLON™, DuPont) offers superb chemical resistance to chlorine, but it exhibits permeability comparable to that of polystyrene, and is a poor barrier if used alone. Particularly suitable barrier films are laminates of two or more of the above materials, such as the PENTAPHARM™ ACLAR™ G03 and G04 laminate films available from Klöckner Pentaplast (Gordonsville, Va.). An internal coating of silica (see PCT patent application publication No. WO 2013/071138), or an external coating of crosslinked vinyl alcohol-vinyl amide copolymer, as described in U.S. Patent application publication No. US 2010/0255326, may be applied to provide enhanced gas barrier properties. Amide-based resins should be regarded as less desirable for the interior surface of laminates, due to the potential for degradation due to formation of N-chloroamides. Polyvinyl alcohol-based resins are likewise less desirable, due to the potential for formation of alkyl hypochlorites. Preferred materials are polyolefins, polyesters, and fluoro- and chloro-olefin polymers that are lacking in chlorine-reactive functional groups.

Metal foil laminates may be employed, provided that the metal is resistant, or can be rendered resistant, to attack by chlorine. Aluminum is almost universally employed in tear-open pouches, due to its low cost, ease of handling, and gas barrier and light-blocking properties, but it is highly reactive toward chlorine. Nonetheless, the inventor has discovered that it is possible to use aluminum in the packaging of the invention, if it is coated with (or deposited on) a particularly effective moisture- and chlorine-barrier polymer. Metals inert to chlorine are few in number; among them are gold, platinum, and palladium. Silver, nickel and tantalum, while they do react with chlorine, develop a protective metal chloride surface coating, and may be considered inert for the purposes of this invention. Methods for laminating gold foil with polymers are known; see for example U.S. Pat. No. 5,702,554. Titanium, tantalum, nickel, and nickel-chromium alloys (NICONEL™) are also suitable, if provided with a protective oxide coating, and are economically more viable than the precious metals, but they are less amenable to vapor deposition.

The degree to which a barrier is “chlorine impermeable” is a function of the permeability of the polymer(s) and other materials in the barrier, the thickness of the layer(s), and the area of the barrier that is exposed to the external atmosphere, relative to the volume of HOCl solution within the container. Also relevant is the amount of chlorine loss from the contained composition that can be tolerated over the desired storage life of the product. As a rough guide for the practitioner, a barrier may be considered “chlorine impermeable” if a composition of the invention, in an unopened package of the invention, loses less than 30% of the contained HOCl over the course of 12 months at room temperature. The effective CTR of the barrier will preferably be less than 10 ml-mil/100 in²-24 hr-atm, and more preferably less than 1 ml-mil/100 in²-24 hr-atm. Most preferably, the CTR will be less than 0.01 ml-mil/100 in²-24 hr-atm.

In certain embodiments of the invention, the compositions may comprise one or more surfactants, in amounts ranging from 0.1% to about 10% by weight, and preferably from about 1% to about 5% by weight, depending on the physicochemical properties (e.g. the critical micelle concentration) and biological properties (e.g., Draize or RBC test results) of the surfactant or surfactant combination. Preferably, the concentration of surfactant(s) will be less than the cmc (critical micelle concentration) in wipes intended for treatment of the eyelids. Surfactants aid in the removal of oils and cellular debris from the skin, giving the active HOCl species more complete access to the surface of the skin. Bacteria that are adherent to the skin and/or to one another, e.g. in a colony or biofilm, can be loosened and suspended by surfactants, resulting in increased exposure to HOCl. Removal of dead skin and other debris from the eyelids also provides a cosmetic improvement to the user.

Surfactants also aid in the wetting of environmental surfaces by the wipes of the invention, thereby improving the level and/or speed of surface disinfection that can be obtained.

Suitable surfactants for a composition of the invention are those stable to hypochlorous acid and chlorine at the pH of the composition. Surfactants usable with HOCl solutions have been described in U.S. Pat. No. 6,793,846, but such compositions are intended for immediate use rather than for storage and later use, and a careful selection from among the disclosed surfactants must be made for use in the present invention. For example, surfactants bearing hydroxyl groups are subject to being converted to alkyl hypochlorites, and are not preferred. Esters and alkyl sulfates are prone to hydrolysis at acidic pH, and are not preferred for compositions having a pH lower than about 5. Surfactants containing amide groups, unless they are tertiary amides, will have NH groups subject to chlorination, and are not preferred. Surfactants bearing alkenyl groups, e.g., unsaturated fatty acid derivatives, are subject to formation of chlorohydrins, and are not preferred. Surfactants relying on weakly acidic groups (e.g. alkyl carboxylates) for hydrophilic affinity will not be preferred at the lower pH range of the present invention, where they may not be fully ionized, although this may be mitigated by the presence of additional surfactant species, or the presence of a quaternary ammonium species on the same molecule (e.g. betaines).

Among the chemically acceptable surfactants, classes that are suitable for use in the present invention include, but are not limited to, alkyl and alkyl ether sulfates, alkyl sulfonates, alkyl sulfoacetates, alkyl sulfobenzoates, quaternary ammonium and imidazolium surfactants, zwitterionic surfactants such as betaines and sulfobetaines, and amine oxide surfactants. Ethoxylated amphiphilic materials may be employed, preferably when appropriately end-capped, e.g. with alkyl ethers, esters, sulfates, sulfoacetates, sulfobenzoates, or sulfoalkyl ethers, such as for example the hypochlorite-resistant polyalkylene oxide non-ionic surfactants disclosed in U.S. Pat. Nos. 4,988,452 and 4,988,462, and the sulfobenzoate-capped anionic surfactants disclosed in U.S. Pat. No. 5,196,133.

For the wipes of the invention that are intended to treat the skin, surfactants that are dermatologically acceptable, i.e. are non-allergenic and non-irritating, are more preferred, and surfactants that do not irritate the eyes will be most preferred for wipes intended to treat blepharitis. Preferred examples are non-ionic and zwitterionic surfactants. Certain surfactants are known to reduce the eye irritation potential of other surfactants, and surfactant combinations may accordingly be used in the present invention. As a general rule, compositions in which the surfactant concentration is below the critical micelle concentration will exhibit less eye irritation, and such concentrations are preferred in compositions of the current invention intended for use on or near the eyes.

As noted above, products specifically designed as eyelid washing compositions for treating blepharitis are available. Simple addition of HOCl to these prior art compositions, however, will not produce a composition of the present invention, due to the incompatibility of numerous components in these prior art products with HOCl and/or chlorine. The prior art surfactants, in particular, contain reactive hydroxyl and amide functional groups.

The invention also provides bandages, preferably adhesive bandages, having an absorbent pad moistened with the compositions of the invention. Such bandages are preferably packaged according to the present invention, in chlorine-resistant and chlorine-impermeable pouches.

The HOCl-moistened wipes of the invention may be used to treat blepharitis, in particular anterior blepharitis, and most particularly anterior blepharitis attributed to S. aureus infection, by gently scrubbing the affected eyelid with a wipe of the invention at intervals of 4 to 12 hours. The wipes may also be used to disinfect the skin prior to insertion of hypodermic needles, cannulas, and the like, by gentle scrubbing. The rapid bactericidal effects of HOCl are particularly valuable in this setting.

Example 1. By way of example, a composition according to the present invention having a pH of 4.0 may be prepared from an aqueous solution of HOCl, 100 ppm (100 mg/liter), having less than 5 ppm chloride, produced as described in U.S. Pat. No. 3,616,385. To 1,000 ml of this solution at room temperature, in a brown bottle or low-actinic flask, is added 3-(decyldimethylammonio) propanesulfonate inner salt (50 g, 5.0% w/v), disodium hydrogen phosphate dodecahydrate (2.98 g) and potassium dihydrogen phosphate (1.13 g), and the mixture is stirred in the dark until a homogeneous solution is obtained. The composition is stored in a brown glass bottle with a polyethylene liner; the inner surface of the liner is covered with a thermally-adhered film of PENTAPHARM™ ACLAR™ G03 laminate (Klöckner Pentaplast, Gordonsville, Va.).

### Example 2

The method of Example 1 is followed, but using as the surfactant sodium lauryl sulfoacetate (25 g, 2.5%).

### Example 3

POWER SHOCK™ 78% Ca(OCl)₂ (5.0 g) is stirred into 1,000 ml of ice cold distilled or deionized (DI) water, and sulfuric or phosphoric acid is added dropwise with stirring until the pH is 5.0 and is holding steady. Solids are removed by filtration or centrifugation. The resulting solution is ca. 53 mM (2.8 g/l, 2800 ppm) in HOCl, and contains at most 0.25 g (7 mM) chloride. An aliquot of DOWEX™ RETARDION™ 11A8 resin (200 ml) is slurried with DI water and transferred to a 3.5-cm diameter glass chromatography column. The resin is rinsed with two column volumes of DI water, and the HOCl solution is then applied to the column and eluted with the aid of a slight positive air pressure. The eluant is analyzed for active chlorine (colorimetric DPD assay), adjusted to pH 4.5-5.0 by addition of 0.5 g sodium acetate and about 0.2 ml acetic acid, and diluted to a working concentration of 500 ppm HOCl (676 mg/l active chlorine.) A liter of resin (1.2 meq/ml) will have the theoretical capacity to desalt 170 liters of this solution, which may then be diluted to ca. 950 liters of a low-chloride, 500 ppm HOCl solution of the present invention.

### Example 4

In order to prepare wiping articles of the invention, a spunbond hydrophilic polyolefin fabric having a basis weight of 1.25 oz/yd² and a fiber denier of 2.4 dpf (Ultrasoft Spunbond, Kimberly-Clark Corp., Roswell, Ga.) is punched into 4.5 cm disks. The disks are assembled into stacks of 60 articles, and each stack is placed in a 120-ml brown glass jar (amber glass 4-oz straight sided jar with polypropylene cap and F217 polyethylene foam liner, item 05-4SSF2241, Greenwood Products, Piscataway N.J.) The inner surface of the cap liner is covered with a thermally-adhered film of PENTAPHARM™ ACLAR™ G03 laminate. The composition of Example 1 is poured into the jars, saturating the disks. The jar is then filled to the brim with the composition, and cap is tightly affixed.

### Example 5

A spunbond hydrophilic polyolefin fabric having a basis weight of 1.25 oz/yd² and a fiber denier of 2.4 dpf (Ultrasoft Spunbond, Kimberly-Clark Corp., Roswell, Ga.) is cut into square pads, 3.5 cm on a side. A laminate of gold foil between two layers of HDPE (0.3 mm, U.S. Pat. No. 5,702,554) is cut into squares, 5 cm on a side. Two rectangles of laminate are laid one over the other, in register, and three sides of the rectangle are thermally sealed with the requisite amount of heat and pressure, the sealing area being about 5 mm all around the perimeter. Into the resulting pouch is placed a square pad, and 2.5 ml of the composition of Example 2 is added by pipette. The pouch is squeezed to bring the liquid level to within 5 mm of the top edge, and the top (fourth) side is thermally sealed, capturing no air inside the resulting pouch. Short slits are cut into opposite sides, 5 mm from one end, as an aid to tearing open the pouch.

### Example 6

To treat blepharitis, a pad or gauze saturated with the composition of Example 1, or a wiping article of Example 2 or Example 5, is lightly squeezed or wrung just to the point that solution does not drip from the article, and with the affected eye closed, the eyelid and eyelashes are gently scrubbed with the wet article for about ten seconds. The skin is allowed to air dry. The treatment is repeated every 8 hours for at least 7 days, preferably 10 days, or for as long as symptoms persist.

By essentially the same method as used in Example 6, other parts of the body may be cleaned and disinfected. To treat acne, for example, or to disinfect the skin in preparation for an injection, the skin is scrubbed by the methods of Example 5. Superficial wounds and burns may be treated by gently wiping with a pad, gauze, or wipe as described above.

The invention also provides pads and gauzes, as described above, incorporated into bandages which may be applied to wounds and burns so as to provide more prolonged exposure of pathogens to the composition of the invention. Deep wounds, including but not limited to diabetic wounds and accidental or combat injuries, as well as serious burns, may be irrigated and cleansed with the compositions of the invention, and dressed with the bandages of the invention.

### Example 7

Cap liners providing a chlorine barrier. A test solution containing roughly 0.2% w/v hypochlorous acid was prepared by combining household sodium hypochlorite bleach (4 parts), 5% aqueous acetic acid (1 part), and distilled water (45 parts). The resulting solution was immediately loaded into 2-oz amber straight-sided glass jars fitted with 53-400 polypropylene caps, provided with cap liners of polypropylene foam, Teflon™ PTFE, or 7.8 mil Aclar™ CTFE (Electron Microscopy Sciences, Hatfield Pa.) All jars were prepared in triplicate.

Each 2-oz jar was placed in an 8-oz amber straight-sided glass jar fitted with a 70-400 PTFE-lined polypropylene cap (all jars from Greenwood Products, Piscataway N.J.), and completely submerged in a chlorine capture solution. A triplicate set of jars, having DI water in place of the chlorine test solution, served as controls.

The chlorine capture solution is adapted from the method of Borges & Reis (S. Borges, B. Reis, J. Automated Meth. Mangagement Chem. 2011, article 463286), and consists of a solution of leuco crystal violet (LCV), 50 μM in pH 4.0 acetate buffer. Elemental chlorine diffusing through the cap of the 2-oz jars oxidizes the colorless leuco dye, generating crystal violet, a stable, highly colored species. The capture solution was monitored for absorbance at 590 nm, the λₘₐₓ of crystal violet, on days 1, 3, 7, 15, 31 and 62. Triplicate results were averaged, normalized to zero absorbance at day 1, and plotted vs. time. The results are shown in FIG. 2. PTFE, which is known for being highly permeable to oxygen, was found to be highly permeable to chlorine as well. Only the ACLAR™ (CTFE) liners effectively retained chlorine within the test jars.

### Example 8

Pouch materials having chlorine resistance. Square sheets of various laminates (Table 2) were folded in half and heat-sealed along two edges. A single 4×4-inch sheet of OPTIWIPE™ 828 hydroentangled polyester fiber wipes (MG Chemicals, Surrey, BC, Canada) was folded into quarters and inserted into each pouch, and all but ⅛ inch of the third side was heat-sealed, leaving a corner gap. The test solution of Example 7 (3 ml) was pipetted into each pouch, and the corner gap was heat-sealed.

Seven pouches were prepared from each laminate to be tested. All pouches were placed in an oven and maintained at 43° C. as an accelerated ageing test. Samples were withdrawn at 2-week intervals, opened, and evaluated for changes in inter-layer adhesion. Changes in package integrity are reported on a scale of 0-100, where 0 represents total separation of the layers and 100 represents full original integrity. In Table 2, “Laminate structure” is an ordered list of the layers making up the pouch membrane, from the outside to the inside. For example, the laminate EFS 145-001 features an exterior surface of printable coated PET polyester. Thicknesses are in mil.

TABLE 2
Chlorine Resistance of Pouch Materials

| | Laminate | Score at | Score at | Score at | Score at |
|---|---|---|---|---|---|
| Laminate ID | structure | Week 2 | Week 4 | Week 6 | Week 8 |
| EFS 145-001 | 0.48 PET | 100 | 85 | 45 | 45 |
| | 0.75 WLDPE | | | | |
| | .285 Al foil | | | | |
| | 0.75 GRX | | | | |
| | 1.25 LLDPE | | | | |
| EFS 317-001 | PET | 0 | 0 | 0 | — |
| | WLDPE | | | | |
| | .285 Al foil | | | | |
| | HPC | | | | |
| | 1.5 EVOH coex | | | | |
| EFS 321-001 | PET | 0 | 0 | — | — |
| | WLDPE | | | | |
| | .285 Al foil | | | | |
| | HPC | | | | |
| | 1.5 nylon coex | | | | |
| ESO 058-001 | PET | 15 | 0 | 0 | 0 |
| | WLDPE | | | | |
| | 2.0 coex HDPE | | | | |
| EFS 278-002 | PET | 15 | 0 | — | — |
| | EAA | | | | |
| | 1.0 Al foil | | | | |
| | EAA | | | | |
| | 1.5 HDPEF | | | | |

PET = polyethylene terephthalate;
WLDPE = white low density polyethylene
GRX = ethylene-acrylic acid tie layer (proprietary, Glenroy Inc.);
HPC = tie layer for foil (proprietary, Glenroy Inc.);
LLDPE = linear low density polyethylene;
EVOH = ethylene-vinyl alcohol copolymer;
HDPE = high density polyethylene;
HDPEF = high density polyethylene, fluorinated;
EAA = ethylene acrylic acid;
coex = coextruded

It is evident from the results shown in Table 2 that chlorine resistance in flexible pouches is not readily attained. Surprisingly, the most resistant pouch material is an aluminum foil-based structure. Aluminum is highly reactive toward chlorine, therefore this result was entirely unexpected. Without being bound by theory, the inventor believes that the LLDPE and/or the ethylene-acrylic acid tie layer prevent water from reaching the metal layer, and that this permits the formation of a protective aluminum chloride surface layer on the foil, analogous to the insoluble silver chloride surface layer created when silver is exposed to chlorine gas. The theory finds support in the fact that corrosion at the cut edges of other foil laminates (not shown) had previously been observed in informal testing.

The test solution contains chlorine at an estimated 100 times the concentration expected in the working HOCl solutions of the invention; furthermore the results shown in Table 2 are from accelerated testing at elevated temperature. The gas barrier properties of metal foils is known to be outstanding. For these reasons, the laminate designated EFS 145-001 is deemed satisfactory, in practice, for the construction of the chlorine-resistant and chlorine-impermeable flexible pouches of the invention.

Those of skill in the art will appreciate that there are obvious variations and modifications to the described invention and the examples provided above, that are within the ability of one of ordinary skill in the art to conceive and carry out. Such obvious variations and modifications are intended to be within the scope of the invention, the exclusive rights to which are limited only by the scope of the following claims.

1. A disinfecting composition, having a pH from 3.0 to 7.0, comprising water, hypochlorous acid, one or more buffers, and a concentration of chloride that is less than about 0.5% w/v.

2. The composition of claim 1, further comprising a surfactant, wherein the surfactant is storage-stable in the presence of hypochlorous acid in the pH range 3.0 to 7.0.

3. The composition of claim 2, wherein the surfactant is a dermatologically-acceptable surfactant.

4. The composition of claim 1, having a pH from 4.0 to 6.0.

5. The composition of claim 2, having a pH from 4.0 to 6.0.

6. The composition of claim 3, having a pH from 4.0 to 6.0.

7. A chlorine-impermeable and chlorine-resistant package, containing a composition according to claim 1.

8. A chlorine-impermeable and chlorine-resistant package, containing a composition according to claim 2.

9. A chlorine-impermeable and chlorine-resistant package, containing a composition according to claim 3.

10-12. (canceled)

13. The chlorine-impermeable and chlorine-resistant package according to claim 7, wherein the package retains substantially zero headspace as the composition is dispensed.

14. The chlorine-impermeable and chlorine-resistant package according to claim 8, wherein the package retains substantially zero headspace as the composition is dispensed.

15. The chlorine-impermeable and chlorine-resistant package according to claim 9, wherein the package retains substantially zero headspace as the composition is dispensed.

16. A disposable wiping article impregnated with a composition according to claim 1.

17. A disposable wiping article impregnated with a composition according to claim 2.

18. A disposable wiping article impregnated with a composition according to claim 3.

19-21. (canceled)

22. A chlorine-impermeable and chlorine-resistant package, containing one or more wiping articles according to claim 16.

23. A chlorine-impermeable and chlorine-resistant package, containing one or more wiping articles according to claim 17.

24. A chlorine-impermeable and chlorine-resistant package, containing one or more wiping articles according to claim 18.

25-36. (canceled)
